(19) United States
(12) Patent Application Publication
(10) Pub. No.: US 2021/0038876 A1
(43) Pub. Date: Feb. 11, 2021

(54) IMPLANTABLE ULTRASOUND CONDUCTING AND DRUG DELIVERING APPARATUS

(71) Applicant: Tse-Ying LIU, Taipei (TW)

(72) Inventors: Tse-Ying LIU, Taipei (TW); Ming-Hong CHEN, Taipei (TW)

(21) Appl. No.: 17/049,743

(22) PCT Filed: Jul. 2, 2018
(86) PCT No.: PCT/CN2018/094082

§ 371 Date: Oct. 22, 2020

(30) Foreign Application Priority Data
Apr. 23, 2018 (CN) 201810368409.7

(51) Int. Cl. A61M 37/00; A61N 7/00

(52) U.S. Cl. CPC A61M 37/0092; A61N 2007/0004; A61N 7/00

(57) ABSTRACT

An implantable ultrasound conducting and drug delivering apparatus includes a drug-accommodating member and a shell-shaped ultrasound-scattering member mounted on a bottom of the drug-accommodating member. The shell-shaped ultrasound-scattering member thereon has a plurality of scattering through-holes. The drug-accommodating member has at least one linking through-hole formed on the bottom thereof to communicate the bottom of the drug-accommodating member with the shell-shaped ultrasound-scattering member. The shell-shaped ultrasound-scattering member is fitted to be disposed within a physical cavity of a patient. A drug is injected into an accommodating room of the drug-accommodating member. The drug passes through the scattering through-holes and delivers to the physical cavity. An ultrasound propagates to the scattering through-holes, and is scattered by the scattering through-holes to the tissue liquid in the physical cavity, all surfaces of an inner wall of the physical cavity and all tissues neighboring the inner wall of the physical cavity.

### CROSS-REFERENCE TO RELATED APPLICATION

This application claims the benefit of International Application No. PCT/CN2018/094082, filed on Jul. 2, 2018, which application is hereby incorporated herein by reference.

### BACKGROUND OF THE INVENTION

### 1. Field of the Invention

The invention relates to an ultrasound conducting and drug delivering apparatus, and in particular, to an ultrasound conducting and drug delivering apparatus which is implanted into a physical cavity of a patient.

For the related technical background of this present invention, please refer to the following references.
[1] Sergio Dromi, Clin Cancer Res 2007, 13(9), p. 2722;
[2] Nikolitsa Nomikou, Acta Biomaterialia 8, 2012, pp. 1273-1280;
[3] Feng-Yi Yang, Journal of Controlled Release 150, 2011, pp. 111-116.

### 2. Description of the Prior Art

Many studies have confirmed that ultrasound can promote the efficacy of drugs. Studies have confirmed that ultrasound can trigger drug carriers to release drugs. For example, ultrasound is used to act with temperature-sensitive liposomes (a drug carrier) to give effective drug release which can be used to treat cancer [1].

There are studies showing that the use of ultrasound can help temporarily open the cell membrane so that genes or gene carriers can enter the cell smoothly. For example, ultrasound is used to fire microbubbles containing genes. The ultrasound and vibration wave of microbubble bursting can temporarily open the cell membrane to allow genes to pass through the barrier of the cell membrane and enter the cell [2]. This phenomenon is called “ultrasound enhanced endocytosis”.

There are studies confirming that the use of ultrasound can help substances to pass through blood vessel walls. Ultrasonic waves can help drug molecules to cross from inside blood vessels to outside blood vessels. This phenomenon is called “ultrasound-enhanced extravasation”. For example, ultrasound is used to assist drugs through the brain blood barrier (BBB) [3]. This barrier is the main barrier for many drugs that cannot be absorbed by brain cells, because hydrophilic drugs will only stay in the blood vessels and cannot cross the blood vessel barriers to the outside of the blood vessels for absorption by brain cells.

However, the ultrasonic application of the above-mentioned prior art utilizes several dispersed ultrasonic sources to emit toward a focal point to form a high-intensity of ultrasound at the focal point. Obviously, the ultrasonic application of the above-mentioned prior art is too complicated to be implemented. If the above-mentioned prior art is used in the treatment of brain diseases, it is necessary to calculate and simulate or use MRI guidance, otherwise it is easy to gather energy in the wrong location and cause irreversible brain damage. Moreover, the above-mentioned prior art cannot be applied to all tissue liquids in the physical cavity of a patient formed due to an operation, nor can the ultrasonic energy be uniformly projected onto all surfaces of an inner wall of the physical cavity and all tissues neighboring the inner wall of the physical cavity.

In addition, the inventors of this present application found that gold nanoparticles coated with porous silica were used with hyaluronic acid as a colloidal stabilizer and 5ALA (which can be effectively accumulate in cancer cells and be specifically transformed to PpIX sono-sensitizer) and combined with ultrasonic sonodynamic therapy and radiotherapy to successfully kill cancer cells with low-dose radiation to protect normal tissues. This provides a precise radiotherapy for deep tumors, and low-dose radiotherapy also greatly reduces side effects.

There is still no apparatus that integrates the two functions of drug delivery and the effective conduction of ultrasonic energy. In addition, this apparatus must effectively conduct unidirectional (directional) ultrasound uniformly to a tissue liquid in a physical cavity of a patient formed due to an operation, all surfaces of an inner wall of the physical cavity, and all tissues neighboring the inner wall of the physical cavity. With this apparatus, there will be no local tissues of the patient that have received too low ultrasound energy to cause low treatment effectiveness, and there will be no local tissues of the patient that receive too high ultrasound energy to cause irreversible tissue damage.

### SUMMARY OF THE INVENTION

Accordingly, one scope of the invention is to provide an implantable ultrasound conducting and drug delivering apparatus. The implantable ultrasound conducting and drug delivering apparatus according to the invention can enhance the efficiency of drug delivery and promote the efficacy of drugs. Moreover, the implantable ultrasound conducting and drug delivering apparatus according to the invention can effectively conduct unidirectionally propagating ultrasound uniformly to a tissue liquid in a physical cavity of a patient formed due to an operation, all surfaces of an inner wall of the physical cavity, and all tissues neighboring the inner wall of the physical cavity. There will be no local tissues of the patient that have received too low ultrasound energy to cause low treatment effectiveness, and there will be no local tissues of the patient that receive too high ultrasound energy to cause irreversible tissue damage.

An implantable ultrasound conducting and drug delivering apparatus according to a first preferred embodiment of the invention includes a drug-accommodating member and a shell-shaped ultrasound-scattering member. The drug-accommodating member has a top, an accommodating room, an opening formed at the top, a bottom and at least one linking through-hole formed on the bottom. The shell-shaped ultrasound-scattering member is mounted on the bottom of the drug-accommodating member, and surrounds and envelopes the bottom of the drug-accommodating member. The bottom of the drug-accommodating member via the at least one linking through-hole communicating with the shell-shaped ultrasound-scattering member. The shell-shaped ultrasound-scattering member thereon has a plurality of scattering through-holes. The shell-shaped ultrasound-scattering member is fitted to be disposed within a physical cavity of a patient. The top of the drug-accommodating member is placed at a mouth of the physical cavity. A drug is injected into the accommodating room of the drug-accommodating member. The drug passes through the at least one linking through-hole and the shell-shaped ultrasound-scattering member, and delivers to the physical cavity through the scattering through-holes of the shell-shaped ultrasound-scattering member. An external ultrasound propagates to the scattering through-holes of the shell-shaped scattering member through the drug-accommodating member, and is scattered by the scattering through-holes of the shell-shaped ultrasound-scattering member to a tissue liquid in the physical cavity, all surfaces of an inner wall of the physical cavity and all tissues neighboring the inner wall of the physical cavity.

In one embodiment, an appearance of the shell-shaped ultrasound-scattering member can exhibit a semi-sphere body, a sphere body, a droplet-shaped body, a cylinder body or other body capable of surrounding and enveloping the bottom of the drug-accommodating member.

Further, the implantable ultrasound conducting and drug delivering apparatus according to the first preferred embodiment of the invention also includes a membrane. The membrane is mounted on the top of the drug-accommodating member to seal the opening at the top of the drug-accommodating member. The drug is injected into the accommodating room of the drug-accommodating member by puncturing the membrane with an injection apparatus. The external ultrasound propagates to the scattering through-holes of the shell-shaped scattering member through the membrane and the drug-accommodating member, and further is scattered by the scattering through-holes of the shell-shaped ultrasound-scattering member to the tissue liquid in the physical cavity, all surfaces of the inner wall of the physical cavity and all tissues neighboring the inner wall of the physical cavity.

Further, the implantable ultrasound conducting and drug delivering apparatus according to the first preferred embodiment of the invention also includes a fitting member. The fitting member includes a bottom plate and a hollow sleeve part. The bottom plate has an outer through-hole. The hollow sleeve part is bonded to a lower surface of the bottom plate and surrounds a circumference of the outer through-hole of the bottom plate. The top of the drug-accommodating member is sleeved or locked into the hollow sleeve part such that the membrane is exposed within the outer through-hole of the bottom plate.

In one embodiment, the shell-shaped ultrasonic-scattering member thereon also has a plurality of through windows. The external ultrasonic propagating to the plurality of through windows continues to propagate forward.

An implantable ultrasound conducting and drug delivering apparatus according to a second preferred embodiment of the invention includes a drug-accommodating member, at least one ultrasound-generating device and a shell-shaped ultrasound-scattering member. The drug-accommodating member has a top, an accommodating room, an opening formed at the top, a bottom and at least one linking through-hole formed on the bottom. The at least one ultrasound-generating device is disposed in the accommodating room of the drug-accommodating member. Each ultrasound-generating device is electrically connected to an external power source respectively. The shell-shaped ultrasound-scattering member is mounted on the bottom of the drug-accommodating member, and surrounds and envelopes the bottom of the drug-accommodating member. The bottom of the drug-accommodating member via the at least one linking through-hole communicating with the shell-shaped ultrasound-scattering member. The shell-shaped ultrasound-scattering member thereon has a plurality of scattering through-holes. The shell-shaped ultrasound-scattering member is fitted to be disposed within a physical cavity of a patient. The top of the drug-accommodating member is placed at a mouth of the physical cavity. A drug is injected into the accommodating room of the drug-accommodating member. The drug passes through the at least one linking through-hole and the shell-shaped ultrasound-scattering member, and delivers to the physical cavity through the scattering through-holes of the shell-shaped ultrasound-scattering member. The at least one ultrasound-generating device is driven by the external power source to generate an ultrasound. The ultrasound propagates to the scattering through-holes of the shell-shaped scattering member through the drug-accommodating member, and is scattered by the scattering through-holes of the shell-shaped ultrasound-scattering member to a tissue liquid in the physical cavity, all surfaces of an inner wall of the physical cavity and all tissues neighboring the inner wall of the physical cavity.

In one embodiment, an appearance of the shell-shaped ultrasound-scattering member can exhibit a semi-sphere body, a sphere body, a droplet-shaped body, a cylinder body or other body capable of surrounding and enveloping the bottom of the drug-accommodating member.

Further, the implantable ultrasound conducting and drug delivering apparatus according to the second preferred embodiment of the invention also includes a membrane. The drug-accommodating member also includes a fitting part extending outward from a circumference of the top of the drug-accommodating member. The membrane is mounted on the fitting part to seal the opening at the top of the drug-accommodating member. The drug is injected into the accommodating room of the drug-accommodating member by puncturing the membrane with an injection apparatus.

Further, the implantable ultrasound conducting and drug delivering apparatus according to the second preferred embodiment of the invention also includes a communication pipe member. The communication pipe member is disposed on the bottom of the drug-accommodating member and penetrates the bottom of the drug-accommodating member. The at least one ultrasound-generating device surrounds the communication pipe member.

In one embodiment, the bottom of the drug-accommodating member extends into the shell-shaped scattering member. Each ultrasonic-generating device is formed as a strip device, and disposed adjacent to the at least one linking through-hole.

Distinguishable from the prior arts, the implantable ultrasound conducting and drug delivering apparatus according to the invention can enhance the efficiency of drug delivery and promote the efficacy of drugs. Moreover, the implantable ultrasound conducting and drug delivering apparatus according to the invention can effectively conduct unidirectionally propagating ultrasound uniformly to a tissue liquid in a physical cavity of a patient formed due to an operation, all surfaces of an inner wall of the physical cavity, and all tissues neighboring the inner wall of the physical cavity. There will be no local tissues of the patient that have received too low ultrasound energy to cause low treatment effectiveness, and there will be no local tissues of the patient that receive too high ultrasound energy to cause irreversible tissue damage.

The advantage and spirit of the invention may be understood by the following recitations together with the appended drawings.

### BRIEF DESCRIPTION OF THE APPENDED DRAWINGS

FIG. 1 is a perspective view of an implantable ultrasound conducting and drug delivering apparatus according to the first preferred embodiment of the invention.

FIG. 2 is a cross sectional view of the implantable ultrasound conducting and drug delivering apparatus taken along the A-A line of FIG. 1.

FIG. 3 is another perspective view of the implantable ultrasound conducting and drug delivering apparatus according to the first preferred embodiment of the invention.

FIG. 4 is a cross sectional view of the implantable ultrasound conducting and drug delivering apparatus taken along the B-B line of FIG. 3.

FIG. 5 is a cross sectional view of a modification of the implantable ultrasound conducting and drug delivering apparatus according to the first preferred embodiment of the invention.

FIG. 6 is a cross sectional view of another modification of the implantable ultrasound conducting and drug delivering apparatus according to the first preferred embodiment of the invention.

FIG. 7 is a cross sectional view of another modification of the implantable ultrasound conducting and drug delivering apparatus according to the first preferred embodiment of the invention.

FIG. 8 is a perspective view of an implantable ultrasound conducting and drug delivering apparatus according to the second preferred embodiment of the invention.

FIG. 9 is a cross sectional view of the implantable ultrasound conducting and drug delivering apparatus taken along the C-C line of FIG. 8.

FIG. 10 is a cross sectional view of a modification of the implantable ultrasound conducting and drug delivering apparatus according to the second preferred embodiment of the invention.

FIG. 11 is a cross sectional view of another modification of the implantable ultrasound conducting and drug delivering apparatus according to the second preferred embodiment of the invention.

FIG. 12 is a diagram showing the test results of maximum energy/minimum energy ratio of the implantable ultrasound conducting and drug delivering apparatus according to the invention with different aperturing rates through the ultrasonic dispersion test.

FIG. 13 is a diagram showing the test results of energy loss rate of the implantable ultrasound conducting and drug delivering apparatus according to the invention with a double-layered aperturing structure and with different aperturing rates.

FIG. 14 is a diagram showing the energy loss rate test result of the implantable ultrasound conducting and drug delivering apparatus according to the invention with a double-layered aperturing structure and a single-layered aperturing structure respectively and with an aperturing rate of 17%.

### DETAILED DESCRIPTION OF THE INVENTION

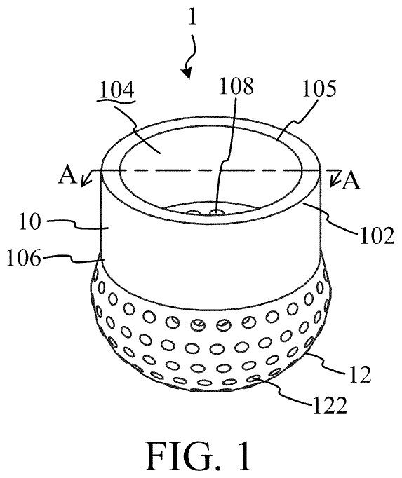

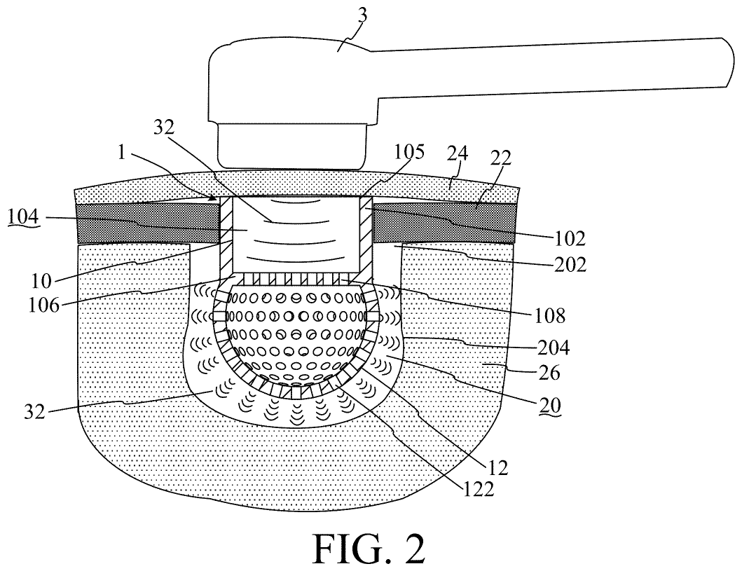

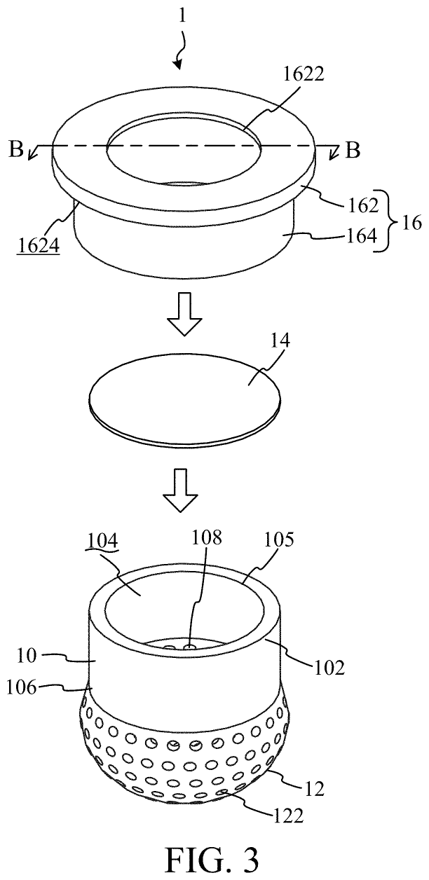

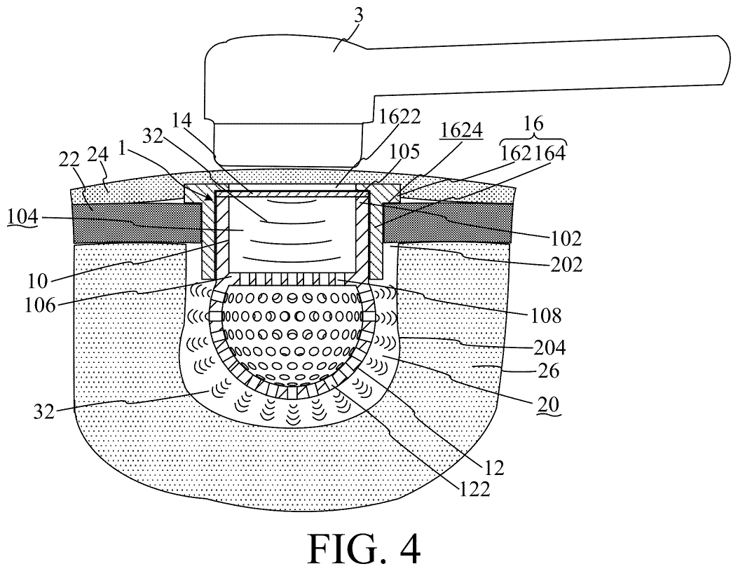

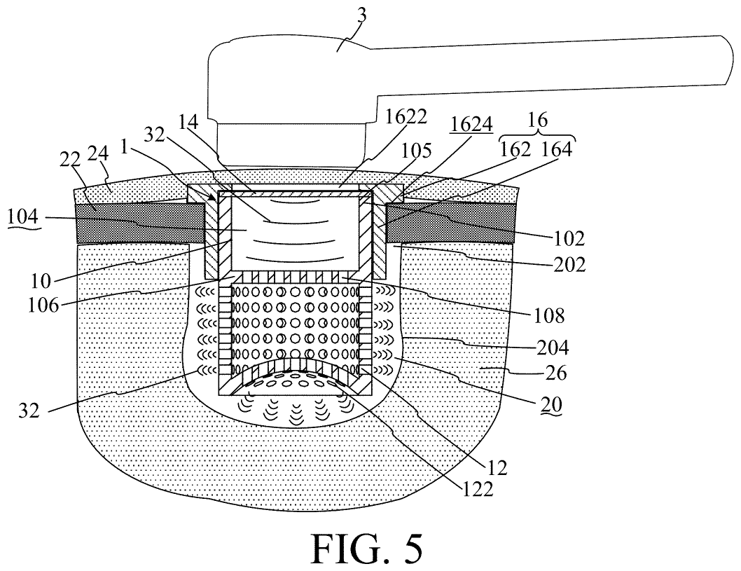

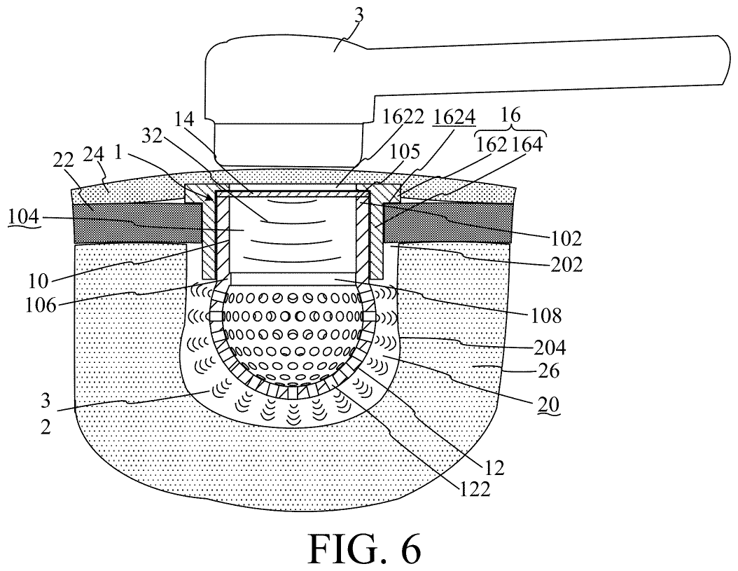

Referring to FIG. 1, FIG. 2, FIG. 3, FIG. 4, FIG. 5 and FIG. 6, those drawings schematically illustrate an implantable ultrasound conducting and drug delivering apparatus 1 according to the first preferred embodiment of the invention. FIGS. 1 and 3 both schematically illustrate with perspective views the implantable ultrasound conducting and drug delivering apparatus 1 according to the first preferred embodiment of the invention. FIG. 2 is a cross sectional view of the implantable ultrasound conducting and drug delivering apparatus 1 taken along the A-A line of FIG. 1. FIG. 4 is a cross sectional view of the implantable ultrasound conducting and drug delivering apparatus 1 taken along the B-B line of FIG. 3. FIG. 5 is a cross sectional view of a modification of the implantable ultrasound conducting and drug delivering apparatus 1 according to the first preferred embodiment of the invention. FIG. 6 is a cross sectional view of another modification of the implantable ultrasound conducting and drug delivering apparatus 1 according to the first preferred embodiment of the invention.

As shown in FIG. 1 and FIG. 2, the implantable ultrasound conducting and drug delivering apparatus 1 according to the first preferred embodiment of the invention includes a drug-accommodating member 10 and a shell-shaped ultrasound-scattering member 12.

Also as shown in FIG. 1 and FIG. 2, the drug-accommodating member 10 has a top 102, an accommodating room 104, an opening 105 formed at the top 102, a bottom 106 and at least one linking through-hole 108 formed on the bottom 106.

Also as shown in FIG. 1 and FIG. 2, the shell-shaped ultrasound-scattering member 12 is mounted on the bottom 106 of the drug-accommodating member 10, and surrounds and envelopes the bottom 106 of the drug-accommodating member 10. The bottom 106 of the drug-accommodating member 10 via the at least one linking through-hole 108 communicating with the shell-shaped ultrasound-scattering member 12. The shell-shaped ultrasound-scattering member 12 thereon has a plurality of scattering through-holes 122.

As shown in FIG. 2, the shell-shaped ultrasound-scattering member 12 is fitted to be disposed within a physical cavity 20 of a patient. The top 102 of the drug-accommodating member 10 is placed at a mouth 202 of the physical cavity 20. In practical applications, the physical cavity 20 of the patient is formed by the patient undergoing surgery, for example, the physical cavity 20 of the patient is formed by the patient after a brain tumor removal operation, as shown in FIG. 2. In FIG. 2, the implantable ultrasound conducting and drug delivering apparatus 1 according to the first preferred embodiment of the invention is placed in the physical cavity 20 through a perforation of the skull 22 of the patient.

In particular, a drug can be injected into the accommodating room 104 of the drug-accommodating member 10. The drug passes through the at least one linking through-hole 108 and the shell-shaped ultrasound-scattering member 12, and delivers to the physical cavity 20 through the scattering through-holes 122 of the shell-shaped ultrasound-scattering member 12. An external ultrasound-generating apparatus 3 generates an external ultrasound 32. The external ultrasound 32 propagates to the scattering through-holes 122 of the shell-shaped ultrasound-scattering member 12 through the drug-accommodating member 10, and is scattered by the scattering through-holes 122 of the shell-shaped ultrasound-scattering member 12 to a tissue liquid in the physical cavity 20, all surfaces of an inner wall 204 of the physical cavity 20 and all tissues 26 neighboring the inner wall 204 of the physical cavity 20. Before the external ultrasound 32 propagates to the scattering through-holes 122 of the shell-shaped ultrasound-scattering member 12, the external ultrasound 32 has been scattered by the at least one linking through-hole 108 in advance.

In practical applications, the tissue liquid of the patient will fill the physical cavity 20 and the implantable ultrasound conducting and drug delivering apparatus 1 according to the first preferred embodiment of the invention. The skin 24 of the patient can be sutured and cover the opening 105 of the drug-accommodating member 10, and thereby, the risk of infection of the patient can be reduced.

In one embodiment, the drug-accommodating member 10 and the shell-shaped ultrasound-scattering member 12 can be integrally formed.

In another embodiment, the drug-accommodating member 10 and the shell-shaped ultrasound-scattering member 12 can be made respectively, and then, the shell-shaped ultrasound-scattering member 12 is mounted on the bottom 106 of the drug-accommodating member 10.

In one embodiment, the drug-accommodating member 10 and the shell-shaped ultrasound-scattering member 12 can be made of ABS, PC, PS, PP, 316L stainless steel, antibacterial stainless steel, titanium alloy, ceramic, etc.

Further, as shown in FIG. 3 and FIG. 4, the implantable ultrasound conducting and drug delivering apparatus 1 according to the first preferred embodiment of the invention also includes a membrane 14. The membrane 14 is mounted on the top 102 of the drug-accommodating member 10 to seal the opening 105 at the top 102 of the drug-accommodating member 10. The drug can be injected into the accommodating room 104 of the drug-accommodating member 10 by puncturing the membrane 14 with an injection apparatus (not shown in FIG. 3 and FIG. 4). The external ultrasound 32 propagates to the scattering through-holes 122 of the shell-shaped ultrasound-scattering member 12 through the membrane 14 and the drug-accommodating member 10, and further is scattered by the scattering through-holes 122 of the shell-shaped ultrasound-scattering member 12 to the tissue liquid in the physical cavity 20, all surfaces of the inner wall 204 of the physical cavity 20 and all tissues 26 neighboring the inner wall 204 of the physical cavity 20. In practical applications, the skin 24 of the patient can be sutured and cover the membrane 14, and thereby, the risk of infection of the patient can be reduced. The components and devices in FIG. 3 and FIG. 4 with the same numerical notations as those in FIG. 1 and FIG. 2 have the same or similar structures and functions, and will be not described in detail herein.

In one embodiment, the membrane 14 can be made of a biocompatible polymer material.

Further, as shown in FIG. 3 and FIG. 4, the implantable ultrasound conducting and drug delivering apparatus 1 according to the first preferred embodiment of the invention also includes a fitting member 16. The fitting member 16 includes a bottom plate 162 and a hollow sleeve part 164. The bottom plate 162 has an outer through-hole 1622. The hollow sleeve part 164 is bonded to a lower surface 1624 of the bottom plate 162 and surrounds a circumference of the outer through-hole 1622 of the bottom plate 162. The top 102 of the drug-accommodating member 10 is sleeved or locked into the hollow sleeve part 164 such that the membrane 14 is exposed within the outer through-hole 1622 of the bottom plate 162. The components and devices in FIG. 3 and FIG. 4 with the same numerical notations as those in FIG. 1 and FIG. 2 have the same or similar structures and functions, and will be not described in detail herein.

In one embodiment, an appearance of the shell-shaped ultrasound-scattering member 12 can exhibit a semi-sphere body (as shown in FIG. 2), a sphere body, a droplet-shaped body, a cylinder body or other body capable of surrounding and enveloping the bottom 106 of the drug-accommodating member 10. Regarding a modification of the implantable ultrasound conducting and drug delivering apparatus 1 according to the first preferred embodiment of the invention, as shown in FIG. 5, the appearance of the shell-shaped ultrasound-scattering member 12 exhibits a cylinder body, and the bottom 106 of the shell-shaped ultrasound-scattering member 12 is recessed inward. The components and devices in FIG. 5 with the same numerical notations as those in FIG. 2 have the same or similar structures and functions, and will be not described in detail herein.

Regarding another modification of the implantable ultrasound conducting and drug delivering apparatus 1 according to the first preferred embodiment of the invention, as shown in FIG. 6, the at least one linking through-hole 108 is a single aperture through the bottom 106 of the drug-accommodating member 10. The components and devices in FIG. 6 with the same numerical notations as those in FIG. 2 have the same or similar structures and functions, and will be not described in detail herein.

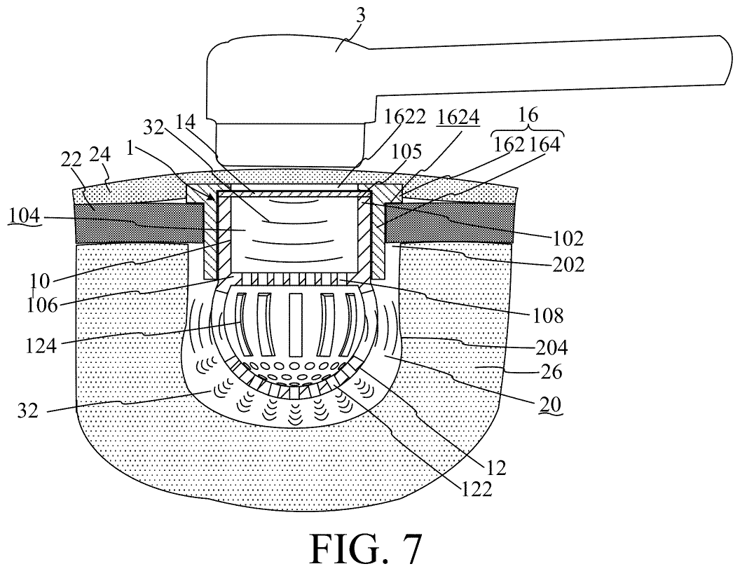

Regarding another modification of the implantable ultrasound conducting and drug delivering apparatus 1 according to the first preferred embodiment of the invention, as shown in FIG. 7, the shell-shaped ultrasonic-scattering member thereon also has a plurality of through windows 124. The external ultrasound 32 propagating to the plurality of through windows 124 continues to propagate forward. The components and devices in FIG. 7 with the same numerical notations as those in FIG. 2 have the same or similar structures and functions, and will be not described in detail herein.

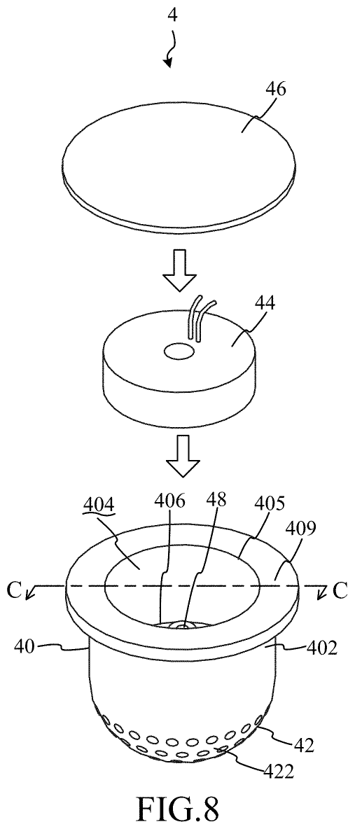

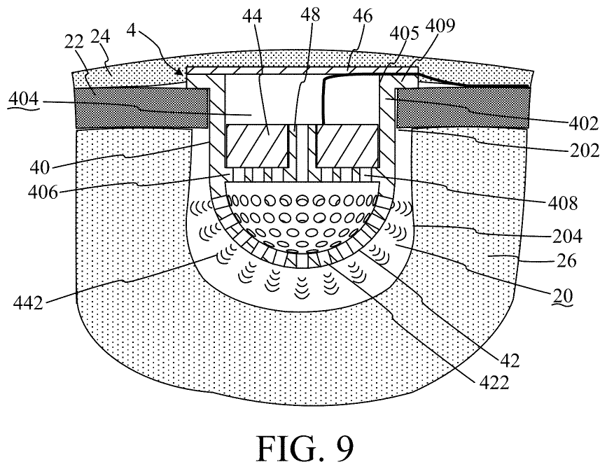

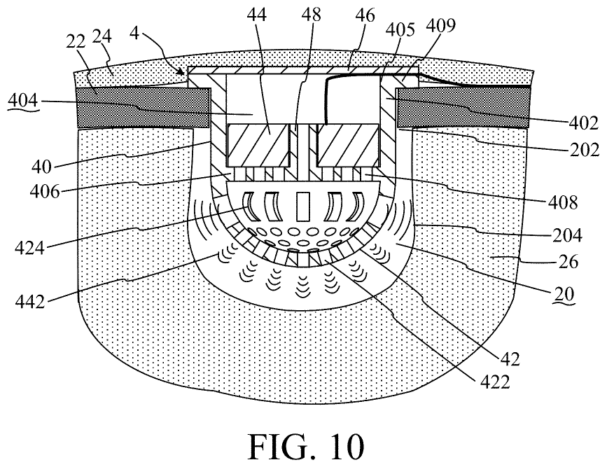

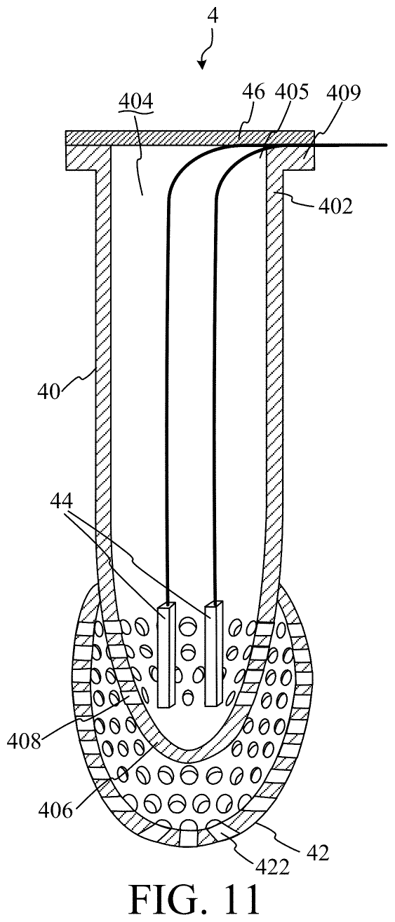

Referring to FIG. 8, FIG. 9 and FIG. 10, those drawings schematically illustrate an implantable ultrasound conducting and drug delivering apparatus 4 according to the second preferred embodiment of the invention. FIG. 8 schematically illustrates with a perspective view the implantable ultrasound conducting and drug delivering apparatus 4 according to the second preferred embodiment of the invention. FIG. 9 is a cross sectional view of the implantable ultrasound conducting and drug delivering apparatus 4 taken along the C-C line of FIG. 8. FIG. 10 is a cross sectional view of a modification of the implantable ultrasound conducting and drug delivering apparatus 4 according to the second preferred embodiment of the invention. FIG. 11 is a cross sectional view of another modification of the implantable ultrasound conducting and drug delivering apparatus 4 according to the second preferred embodiment of the invention.

As shown in FIG. 8 and FIG. 9, the implantable ultrasound conducting and drug delivering apparatus 4 according to the second preferred embodiment of the invention includes a drug-accommodating member 40, at least one ultrasound-generating device 44 and a shell-shaped ultrasound-scattering member 42. In FIG. 8, the at least one ultrasound-generating device 44 is, but not limited to, a single ring device.

Also as shown in FIG. 8 and FIG. 9, the drug-accommodating member 40 has a top 402, an accommodating room 404, an opening 405 formed at the top 402, a bottom 406 and at least one linking through-hole 408 formed on the bottom 406.

Also as shown in FIG. 8 and FIG. 9, the at least one ultrasound-generating device 44 is disposed in the accommodating room 404 of the drug-accommodating member 40. Each ultrasound-generating device 44 is electrically connected to an external power source respectively.

Also as shown in FIG. 8 and FIG. 9, the shell-shaped ultrasound-scattering member 42 is mounted on the bottom 406 of the drug-accommodating member 40, and surrounds and envelopes the bottom 406 of the drug-accommodating member 40. The bottom 406 of the drug-accommodating member 40 via the at least one linking through-hole 408 communicating with the shell-shaped ultrasound-scattering member 42. The shell-shaped ultrasound-scattering member 42 thereon has a plurality of scattering through-holes 422.

As shown in FIG. 9, the shell-shaped ultrasound-scattering member 42 is fitted to be disposed within a physical cavity 20 of a patient. The top 402 of the drug-accommodating member 40 is placed at a mouth 202 of the physical cavity 20. A drug can be injected into the accommodating room 404 of the drug-accommodating member 40. The drug passes through the at least one linking through-hole 408 and the shell-shaped ultrasound-scattering member 42, and delivers to the physical cavity 20 through the scattering through-holes 422 of the shell-shaped ultrasound-scattering member 42. The at least one ultrasound-generating device 44 can be driven by the external power source to generate an ultrasound 442. The ultrasound 442 propagates to the scattering through-holes 422 of the shell-shaped scattering member through the drug-accommodating member 40, and is scattered by the scattering through-holes 422 of the shell-shaped ultrasound-scattering member 42 to a tissue liquid in the physical cavity 20, all surfaces of an inner wall 204 of the physical cavity 20 and all tissues neighboring the inner wall 204 of the physical cavity 20. Before the ultrasound 442 propagates to the scattering through-holes 422 of the shell-shaped ultrasound-scattering member 42, the ultrasound 442 has been scattered by the at least one linking through-hole 408 in advance.

In practical applications, the tissue liquid of the patient will fill the physical cavity 20 and the implantable ultrasound conducting and drug delivering apparatus 4 according to the second preferred embodiment of the invention. The skin 24 of the patient can be sutured and cover the opening 405 of the drug-accommodating member 40, and thereby, the risk of infection of the patient can be reduced.

In one embodiment, the external power source can be a rechargeable battery. The rechargeable battery can be placed away from the physical cavity 20. The wire connecting the at least one ultrasound-generating device 44 and the rechargeable battery can be placed under the patient's skin 24, and thereby, the risk of infection of the patient can be reduced. The rechargeable battery can be wirelessly charged by a coil, and thereby, the rechargeable battery can be prevented from contacting external pollution source.

In one embodiment, the drug-accommodating member 40 and the shell-shaped ultrasound-scattering member 42 can be integrally formed.

In another embodiment, the drug-accommodating member 40 and the shell-shaped ultrasound-scattering member 42 can be formed respectively, and then, the shell-shaped ultrasound-scattering member 42 is mounted on the bottom 406 of the drug-accommodating member 40.

In one embodiment, the drug-accommodating member 40 and the shell-shaped ultrasound-scattering member 42 can be made of ABS, PC, PS, PP, 316L stainless steel, antibacterial stainless steel, titanium alloy, ceramic, etc.

In one embodiment, each ultrasound-generating device 44 can be, but not limited to, a piezoelectric ceramic device.

Further, as shown in FIG. 8 and FIG. 9, the implantable ultrasound conducting and drug delivering apparatus 4 according to the second preferred embodiment of the invention also includes a membrane 46. The drug-accommodating member 40 also includes a fitting part 409 extending outward from a circumference of the top 402 of the drug-accommodating member 40. The membrane 46 is mounted on the fitting part 409 to seal the opening 405 at the top 402 of the drug-accommodating member 40. The drug can be injected into the accommodating room 404 of the drug-accommodating member 40 by puncturing the membrane 46 with an injection apparatus (not shown in FIG. 8 and FIG. 9). In practical applications, the skin 24 of the patient can be sutured and cover the membrane 46, and thereby, the risk of infection of the patient can be reduced.

In one embodiment, the membrane 46 can be made of a biocompatible polymer material.

Further, as shown in FIG. 8 and FIG. 9, the implantable ultrasound conducting and drug delivering apparatus 4 according to the second preferred embodiment of the invention also includes a communication pipe member 48. The communication pipe member 48 is disposed on the bottom 406 of the drug-accommodating member 40 and penetrates the bottom 406 of the drug-accommodating member 40. The at least one ultrasound-generating device 44 surrounds the communication pipe member 48. In FIG. 8 and FIG. 9 the at least one ultrasound-generating device 44 is formed as a single ring device.

In one embodiment, an appearance of the shell-shaped ultrasound-scattering member 42 can exhibit a semi-sphere body, a sphere body, a droplet-shaped body, a cylinder body or other body capable of surrounding and enveloping the bottom 406 of the drug-accommodating member 40.

Regarding a modification of the implantable ultrasound conducting and drug delivering apparatus 4 according to the second preferred embodiment of the invention, as shown in FIG. 10, the shell-shaped ultrasound-scattering member 42 thereon also has a plurality of through windows 424. The ultrasonic 442 propagating to the plurality of through windows 424 continues to propagate forward.

Regarding another modification of the implantable ultrasound conducting and drug delivering apparatus 4 according to the second preferred embodiment of the invention, as shown in FIG. 11, the bottom 406 of the drug-accommodating member 40 extends into the shell-shaped ultrasound-scattering member 42. Each ultrasound-generating device 44 is formed as a strip device, and disposed adjacent to the at least one linking through-hole 408 of the drug-accommodating member 40. The implantable ultrasound conducting and drug delivering apparatus 44 shown in FIG. 11 is suitable for being implanted in the physical cavity 20 of the patient having a long and narrow channel.

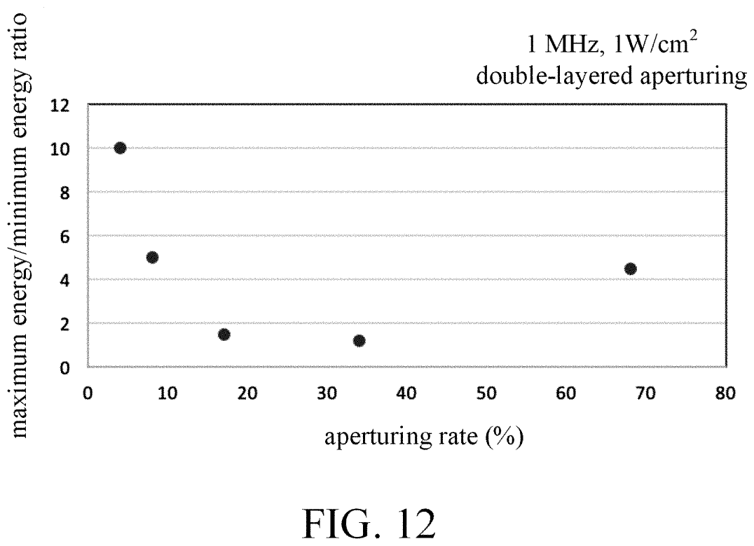

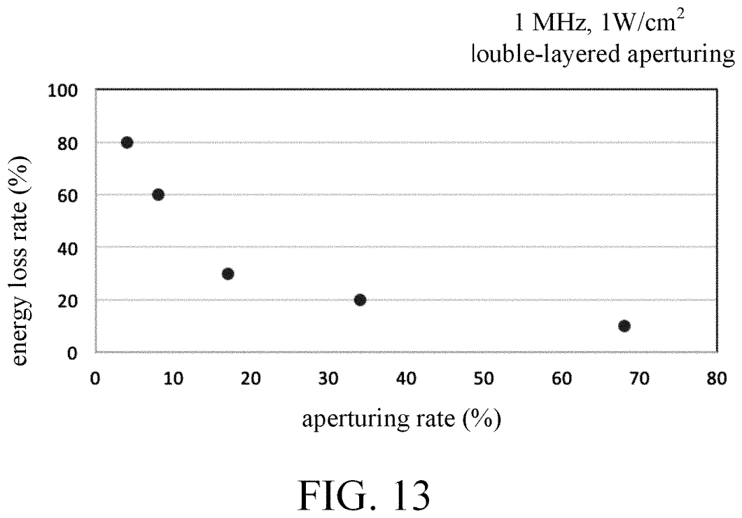

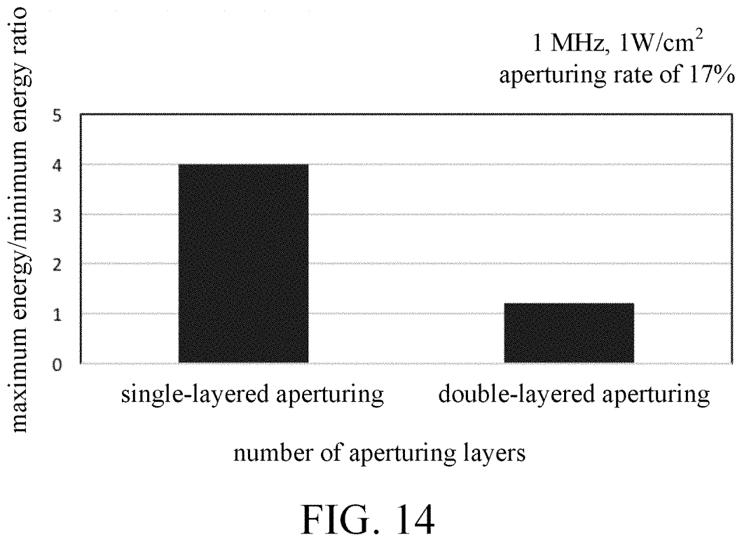

Referring to FIG. 12, FIG. 13 and FIG. 14, FIG. 12 is a diagram showing the test results of maximum energy/minimum energy ratio of the implantable ultrasound conducting and drug delivering apparatus according to the invention with different aperturing rates through the ultrasonic dispersion test. The implantable ultrasound conducting and drug delivering apparatus, undergoing the ultrasonic dispersion test, has a double-layered aperturing structure, that is to say, the implantable ultrasound conducting and drug delivering apparatus has the linking through-holes and the scattering through-holes. FIG. 13 is a diagram showing the test results of energy loss rate of the implantable ultrasound conducting and drug delivering apparatus according to the invention with a double-layered aperturing structure and with different aperturing rates. FIG. 14 is a diagram showing the energy loss rate test result of the implantable ultrasound conducting and drug delivering apparatus according to the invention with a double-layered aperturing structure and a single-layered aperturing structure (only with the scattering through-holes) respectively and with an aperturing rate of 17%. The energy density of the incident ultrasound is 1 W/cm², and the frequency of the incident ultrasound is 1 MHz.

FIG. 12 and FIG. 13 confirm that as the aperturing rate is in the range of from 17% to 34%, the maximum energy/minimum energy ratio closest to 1 can be obtained at a lower energy consumption rate, which means that the ultrasonic dispersion effect is good. As the aperturing rate is less than 17%, the energy consumption rate will be greatly increased. Because it is necessary to consider the structural strength of the implantable ultrasound conducting and drug delivering apparatus according to the invention, it is preferable that the implantable ultrasound conducting and drug delivering apparatus according to the invention adopts, but no limited to, the aperturing rate of 17%. FIG. 14 confirms that the maximum energy/minimum energy ratio of the implantable ultrasound conducting and drug delivering apparatus with the double-layered aperturing structure is much closer to 1, that is to say, the ultrasonic dispersion effect of the implantable ultrasound conducting and drug delivering apparatus with the double-layered aperturing structure is better, and the implantable ultrasound conducting and drug delivering apparatus with the double-layered aperturing structure can avoid local irreversible tissue damage caused by excessive local ultrasound energy.

The advantages of the implantable ultrasound conducting and drug delivering apparatus according to the invention are listed below.

Firstly, with the implantable ultrasound conducting and drug delivering apparatus according to the invention, ultrasound energy does not need to pass through the skull of the patient, so low-energy (low biological effect) ultrasound can be used, which is better controlled and will not cause irreversible brain damage of the patient.

Secondly, with the implantable ultrasound conducting and drug delivering apparatus according to the invention, the drug does not need to penetrate the blood-brain barrier, and can be directly administered through the skull. For cancer cells in or near the space left due to the removal of brain cancer tissues, the drug delivery efficiency (ultra-low dosage without the need of systemic dilution and liver metabolism) are relatively high, and ultrasound-enhanced endocytosis can also help cancer cells to swallow drugs.

Thirdly, with the implantable ultrasound conducting and drug delivering apparatus according to the invention, originally unidirectionally propagating ultrasound will be uniformly scattered toward the three-dimensional space (three-dimensional spherical space). It will not cause irreversible damage to the brain tissue directly in front of the ultrasound-generating device and hard receiving of ultrasound energy of the lateral tissues.

Fourthly, with the implantable ultrasound conducting and drug delivering apparatus according to the invention, the uniformly scattered ultrasound in the physical cavity will also make the drugs transported to the brain through the blood vessels pass through the blood vessels and enter the cancer cells smoothly by the ultrasound-enhanced extravasation.

Fifthly, the implantable ultrasound conducting and drug delivering apparatus according to the invention is easy to operate, so that the correct ultrasound energy and the correct drug concentration can interact with the right (target) location in the brain, and there is no need to calculate or measure whether oral drugs or intravenous drugs in the site of action of the brain has accumulated to a curative concentration or not.

Sixthly, the prior art of focused transcranial ultrasound requires computational simulation or MRI guidance, otherwise it is easy to gather energy in the wrong location of the patient and cause irreversible brain damage of the patient. On the contrary, by operating the implantable ultrasound conducting and drug delivering apparatus according to the invention, irreversible brain damage of the patient caused by ultrasound energy can be avoided.

Seventhly, the implantable ultrasound conducting and drug delivering apparatus according to the invention can allow the medicine to be uniformly mixed in the apparatus, and then gradually diffuse out, and not only to the tissue near the through holes.

Eighthly, by using the implantable ultrasound conducting and drug delivering apparatus according to the invention, the ultrasound-generating device does not need to be frequently percutaneously entered into the body of the patient, and frequent aseptic operations can be eliminated, and the chance of infection of the patient when operating the ultrasound-generating device is reduced to zero. The implantable ultrasound conducting and drug delivering apparatus according to the invention is a subcutaneous implant rather than a penetrating implant, and there is no interface and channel through which bacteria can enter the body of the patient. The implantable ultrasound conducting and drug delivering apparatus according to the invention can be implanted for more than one month for a long time, so that the patient can frequently use ultrasound devices for a long time without any concern about infection.

With the embodiment and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

1. An implantable ultrasound conducting and drug delivering apparatus, comprising:
a drug-accommodating member, having a top, an accommodating room, an opening formed at the top, a bottom and at least one linking through-hole formed on the bottom; and
a shell-shaped ultrasound-scattering member, being mounted on the bottom of the drug-accommodating member, and surrounding and enveloping the bottom of the drug-accommodating member, the bottom of the drug-accommodating member via the at least one linking through-hole communicating with the shell-shaped ultrasound-scattering member, the shell-shaped ultrasound-scattering member thereon having a plurality of scattering through-holes, wherein the shell-shaped ultrasound-scattering member is fitted to be disposed within a physical cavity of a patient, and the top of the drug-accommodating member is placed at a mouth of the physical cavity, wherein an appearance of the shell-shaped ultrasound-scattering member exhibits one selected from the group consisting of a semi-sphere body, a sphere body, a droplet-shaped body and a cylinder body;
wherein a drug is injected into the accommodating room of the drug-accommodating member, the drug passes through the at least one linking through-hole and the shell-shaped ultrasound-scattering member, and delivers to the physical cavity through the scattering through-holes, an external ultrasound propagates to the scattering through-holes of the shell-shaped scattering member through the drug-accommodating member, and is scattered by the scattering through-holes of the shell-shaped ultrasound-scattering member to a tissue liquid in the physical cavity, all surfaces of an inner wall of the physical cavity and all tissues neighboring the inner wall of the physical cavity.

2. The implantable ultrasound conducting and drug delivering apparatus of claim 1, further comprising a membrane, mounted on the top of the drug-accommodating member to seal the opening, wherein the drug is injected into the accommodating room of the drug-accommodating member by puncturing the membrane with an injection apparatus, the external ultrasound propagates to the scattering through-holes of the shell-shaped scattering member through the membrane and the drug-accommodating member.

3. The implantable ultrasound conducting and drug delivering apparatus of claim 2, further comprising a fitting member, comprising a bottom plate and a hollow sleeve part, the bottom plate having an outer through-hole, the hollow sleeve part being bonded to a lower surface of the bottom plate and surrounding a circumference of the outer through-hole, the top of the drug-accommodating member is sleeved or locked into the hollow sleeve part such that the membrane is exposed within the outer through-hole.

4. (canceled)

5. The implantable ultrasound conducting and drug delivering apparatus of claim 2, wherein the shell-shaped ultrasonic-scattering member thereon also has a plurality of through windows, and the external ultrasonic propagating to the plurality of through windows continues to propagate forward.

6. An implantable ultrasound conducting and drug delivering apparatus, comprising:
a drug-accommodating member, having a top, an accommodating room, an opening formed at the top, a bottom and at least one linking through-hole formed on the bottom;
at least one ultrasound-generating device, disposed in the accommodating room of the drug-accommodating member, each ultrasound-generating device being electrically connected to an external power source respectively; and
a shell-shaped ultrasound-scattering member, being mounted on the bottom of the drug-accommodating member, and surrounding and enveloping the bottom of the drug-accommodating member, the bottom of the drug-accommodating member via the at least one linking through-hole communicating with the shell-shaped ultrasound-scattering member, the shell-shaped ultrasound-scattering member thereon having a plurality of scattering through-holes, wherein the shell-shaped ultrasound-scattering member is fitted to be disposed within a physical cavity of a patient, and the top of the drug-accommodating member is placed at a mouth of the physical cavity, wherein an appearance of the shell-shaped ultrasound-scattering member exhibits one selected from the group consisting of a semi-sphere body, a sphere body, a droplet-shaped body and a cylinder body;
wherein a drug is injected into the accommodating room of the drug-accommodating member, the drug passes through the at least one linking through-hole and the shell-shaped ultrasound-scattering member, and delivers to the physical cavity through the scattering through-holes, the at least one ultrasound-generating device is driven by the external power source to generate an ultrasound, the ultrasound propagates to the scattering through-holes of the shell-shaped scattering member through the drug-accommodating member, and is scattered by the scattering through-holes of the shell-shaped ultrasound-scattering member to a tissue liquid in the physical cavity, all surfaces of an inner wall of the physical cavity and all tissues neighboring the inner wall of the physical cavity.

7. The implantable ultrasound conducting and drug delivering apparatus of claim 6, further comprising a membrane, wherein the drug-accommodating member also comprises a fitting part extending outward from a circumference of the top of the drug-accommodating member, the membrane is mounted on the fitting part to seal the opening, the drug is injected into the accommodating room of the drug-accommodating member by puncturing the membrane with an injection apparatus.

8. The implantable ultrasound conducting and drug delivering apparatus of claim 7, wherein the bottom of the drug-accommodating member extends into the shell-shaped scattering member, each ultrasonic-generating device is formed as a strip device and disposed adjacent to the at least one linking through-hole.

9. (canceled)

10. The implantable ultrasound conducting and drug delivering apparatus of claim 7, further comprising a communication pipe member, being disposed on the bottom of the drug-accommodating member and penetrating the bottom of the drug-accommodating member, wherein the at least one ultrasound-generating device surrounds the communication pipe member.

11. The implantable ultrasound conducting and drug delivering apparatus of claim 7, wherein the shell-shaped ultrasonic-scattering member thereon also has a plurality of through windows, and the ultrasonic propagating to the plurality of through windows continues to propagate forward.

12. The implantable ultrasound conducting and drug delivering apparatus of claim 2, wherein the at least one linking through-hole is a single aperture through all of the bottom of the drug-accommodating member.
